(19) United States
(12) Patent Application Publication
(10) Pub. No.: US 2014/0256987 A1
(43) Pub. Date: Sep. 11, 2014

(54) Creatinol O-phosphate and synthesis method thereof

(71) Applicant: Guoji ZHANG, Yangliuging (CN)

(72) Inventor: Guoji ZHANG, Yangliuging (CN)

(73) Assignee: Tianjin Tiancheng Pharmaceutical Co., Ltd.

(21) Appl. No.: 13/815,531

(22) Filed: Mar. 9, 2013

(51) Int. Cl. C07C 277/00

(52) U.S. Cl. CPC C07C 277/00
USPC 564/241

(57) ABSTRACT

A creatinol O-phosphate is formed by chemically reacting a creatinol sulfate of creatinol compound with sulfuric acid to form a protected creatinol sulfate as a transition state of the synthesis process of the creatinol O-phosphate, wherein the protected creatinol is further reacted with one of P₂O₅, CIPO₃H₂, and POCl₃ to form the creatinol O-phosphate. The method of producing the protected creatinol sulfate includes a step of adding sulfuric acid into a creatinol sulfate to form a protected creatinol sulfate as the protected functional group. Therefore, the creatinol O-phosphate is formed by the process including the steps of chemically reacting the protected creatinol sulfate with one of POCl₃, ClPO₃OH₂, and P₂O₅ to form a creatinol O-phosphate solution; and crystallizing the creatinol O-phosphate solution to obtain crystallized creatinol O-phosphate.

### CROSS REFERENCE OF RELATED APPLICATION

This is a Divisional application of a non-provisional application having an application Ser. No. 12/590,140 and a filing date of Nov. 2, 2009.

### BACKGROUND OF THE PRESENT INVENTION

1. Field of Invention

The present invention relates to the chemical compounds of creatinol O-phosphate, and more particularly to a method for synthesizing the creatinol O-phosphate.

2. Description of Related Arts

There are lots of chemical compounds are developed for increasing the durability of human muscle. For example, the creatine phosphate is well known for storing energy in muscles, prolonging the durability of muscle, and delaying the muscle fatigue occurring. The creatine phosphate is strongly involved into the process of synthesizing ATP in muscles, which is playing the role of transporting chemical energy within cells for metabolism. When a human being, such as an athlete, is doing exercise, the ATP in muscles is gradually reducing to converting and providing energy to the muscles. The creatine phosphate is able to convert ADP in muscles into ATP, so that the muscle is able to recover relatively sooner.

Generally, the creatine phosphate converts the ADP in muscles to ATP by donating the phosphate group of the creatine phosphate, so that during the intense activity of the human body, the creatine phosphate plays an important role for quickly regenerating the ATP from ADP, so as to reserve the energy of the muscle.

Recently, the researches show that the creatinol O-phosphate, which is acting similar to the creatine phosphate to donate the phosphate group for converting ADP to ATP in muscles, can supply the energy to muscles more effectively than the creatine phosphate. Therefore, the creatinol O-phosphate is able to reserve relatively more energy in muscles, enhance the muscle strength and the explosive force, grow more muscles, increase the flexibility of the muscle, minimize the damage of the muscle, and delay the muscle fatigue.

There are two main methods for synthesizing the creatinol O-phosphate found in the existing techniques. One is to react thiourea with the dibromoethane to obtain the S-ethyl isothiourea hydrobromide, and then to chemically react the N-methyl-amino-ethanol with S-ethyl isothiourea hydrobromide to form creatinol hydrobromide. The creatinol hydrobromide then is further reacted with excess phosphides for phosphorylation to form the creatinol O-phosphate. The yield rate in the above process is about 44.67%. The creatinol O-phosphate is further purified via barium carbonate and barium hydroxide. However, the raw material, thiourea and dibromoethane, of this synthesis method are high in cost. The process for preparing the creatinol hydrobromide also produces the side product of ethanethiol, which is toxic and harmful to the human body and the environment. Due to the excess amount of phosphides, a great amount of HCl gaseous is emitted while treating the waste of the excess phosphides, and corroding the equipment. The large amount of acetone used as a solvent for purification is another complicated issue of waste treatment.

The other way for synthesizing the creatinol O-phosphate is from creatinol.H₃PO₄, wherein the creatinol.H₃PO₄ is heated to a temperature higher than its melting point, and the pressure is reduced (165° C./5 mmHg), so that a 76% yield rate of the creatinol O-phosphate is obtained according to the prior publication and patent. However, according to the publication and patent, the creatinol hydrobromide is chemically reacted with the ethanol aqueous of NaOH having the equal mol as the creatinol hydrobromide to form the free radical of the creatinol O-phosphate. The free radical of the creatinol O-phosphate is unstable in the alkaline solution, so that it might be easily cause dangers if any operator is careless during operating the synthesis process. In addition, the greater toxicity and higher cost of thiourea and dibromoethane are still inevitably used in the synthesis process, and the yield rate is about the same as the first method mentioned above.

### SUMMARY OF THE PRESENT INVENTION

An object of the present invention is to provide a method for synthesizing the chemical compound of creatinol O-phosphate, which has relatively higher purity, so as to reduce the dose for administration to minimize any side effects.

Another object of the present invention is to provide a method for synthesizing the chemical compound of creatinol O-phosphate, wherein a protected creatinol sulfate is formed as a transitional state of the synthesizing process for the chemical compound of creatinol O-phosphate.

Another object of the present invention is to provide a method for synthesizing the chemical compound of creatinol O-phosphate, wherein a protected functional group can reduce the formation of N-substituted impurities, increase reaction selectivity to form the target product, enhance the yield of the target product, and increase the efficiency to produce high purity target product.

Another object of the present invention is to provide a chemical compound of creatinol O-phosphate, which is synthesizing via a creatinol compound containing a creatinol group to react with a chemical compound selected from the group consisting of POCl₃, ClPO₃OH₂, and P₂O₅.

Another object of the present invention is to provide a chemical compound of creatinol O-phosphate, which is able to effectively supply energy to the muscles of organic body while minimizing the dose of administration of the chemical compound.

Another object of the present invention is to provide a creatinol sulfate, wherein the sulfuric acid for forming the protected functional group is interchangeable with HCl, HNO₃, HBr, H₃PO₄, and other acidic compounds.

Accordingly, in order to accomplish the above objects, the present invention provides a method of producing a transitional compound for a synthesizing process of creatinol O-phosphate, comprising the step of adding sulfuric acid into a creatinol compound to form a protected functional group as a transition state of the synthesizing process of creatinol O-phosphate, wherein the protected functional group is adapted for chemically reacting with a chemical compound selected from the group consisting of POCl₃, ClPO₃OH₂, and P₂O₅ to form the creatinol O-phosphate solution.

The present invention further provides a synthesizing process of creatinol O-phosphate, comprising the steps of:

(a) forming a protected functional group as a transition state by adding sulfuric acid into a creatinol compound;

(b) chemically reacting the protected functional group with a chemical compound selected from the group consisting of POCl₃, ClPO₃OH₂, and P₂O₅ to form a creatinol O-phosphate solution; and

(c) crystallizing the creatinol O-phosphate solution to obtain crystallized creatinol O-phosphate.

These and other objectives, features, and advantages of the present invention will become apparent from the following detailed description, the accompanying drawings, and the appended claims.

### BRIEF DESCRIPTION OF THE DRAWINGS

FIG. 1 is a flow chart of a process of synthesizing creatinol O-phosphate according to a preferred embodiment of the present invention.

### DETAILED DESCRIPTION OF THE PREFERRED EMBODIMENT

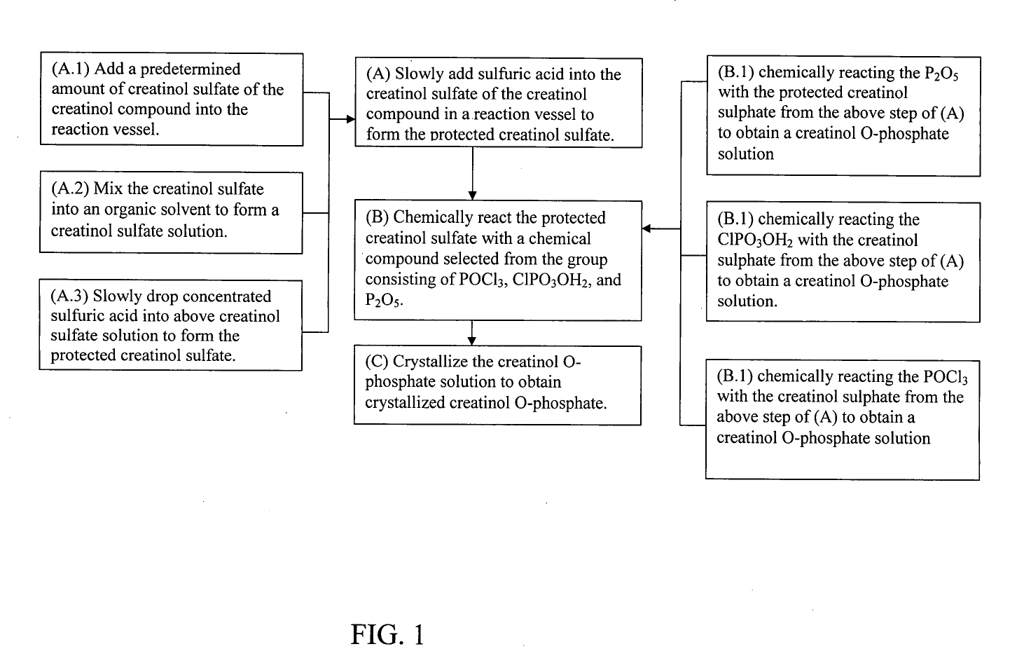

Referring to FIG. 1, a process for synthesizing an organic compound of creatinol O-phosphate is illustrated, wherein the creatinol O-phosphate has a chemical structure of:

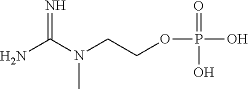

The creatinol O-phosphate is preferably formed from a creatinol compound, which contains a creatinol group chemically reacting with a chemical compound selected from a group consisting of POCl₃, ClPO₃OH₂, and P₂O₅. As one skilled in the art will readily appreciated that the creatinol O-phosphate is able to quickly supply energy to the muscle cells by immediately converting the ADP, which is dramatically reduced and decomposed while doing intense exercise, into ATP, so as to re-store energy to the muscle and delay the fatigue of muscles.

According to the preferred embodiment, the synthesizing process of creatinol O-phosphate comprises the following steps.

(1) Form the protected functional group as a transition state by adding sulfuric acid into a creatinol compound.

(2) Chemically react the protected functional group with the chemical compound selected from the group consisting of POCl₃, ClPO₃OH₂, and P₂O₅ to form a creatinol O-phosphate solution.

(3) Crystallize the creatinol O-phosphate solution to obtain crystallized creatinol O-phosphate.

The creatinol group has a chemical structure as follows:

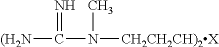

According to the preferred embodiment, the present invention provides the transition state for synthesizing process of creatinol O-phosphate, wherein the protected functional group can reduce the formation of N-substituted impurities, increase reaction selectivity to form the target product, enhance the yield of the target product, and increase the efficiency to produce high purity target product. Accordingly, at the transition state, the protected functional group as the transitional compound is formed by the following steps.

(i) Mix the creatinol compound into an organic solvent to form a creatinol compound solution.

(ii) Slowly drop the sulfuric acid into the creatinol compound solution to form the protected functional group.

Accordingly, the organic solvent in the step (i) is selected from the group consisting of butanone, ethyl acetate, acetone, and other ketones.

In the presently preferred embodiment, the creatinol compound having the creatinol group is preferably a creatinol sulfate, wherein the creatinol sulfate is preferably formed from N-methyl-amino-ethanol chemically reacting with cyanamide and concentrated sulfuric acid to form the creatinol sulfate of the creatinol compound, which contains the creatinol group.

Accordingly, creatinol sulfate is preferably provided for the process of synthesize the creatinol O-phosphate as a transitional compound to form the protected creatinol sulfate, wherein the creatinol O-phosphate is formed by chemically reacting a protected creatinol sulfate with the group consisting of POCl₃, ClPO₃OH₂, and P₂O₅. It is appreciated that the creatinol sulfate may be formed by any method or process for forming the creatinol O-phosphate. Using the relatively cheaper creatinol sulfate not only minimize the manufacturing cost, but also produces relatively less toxicity during the reaction process.

The protected creatinol sulfate is preferably formed by chemically reacting the creatinol sulfate with concentrated sulfuric acid, so that the protected creatinol sulfate as the protected functional group is able to further react with the POCl₃, ClPO₃OH₂, or P₂O₅ to form the creatinol O-phosphate. As will be readily appreciated by one skilled in the art, the creatinol sulfate may also chemically reacts with HCl, HNO₃, HBr, H₃PO₄, sulfonate of alkyl benzene, or other acidic compounds to form the protected functional group, so as to further react with POCl₃, ClPO₃OH₂, or P₂O₅ to form the creatinol O-phosphate. The reaction equation of the creatinol sulfate reacting with the concentrated sulfuric acid to form the protected creatinol sulfate is illustrated as below.

It is worth to mention that the creatinol O-phosphate may be formed from other creatinol compounds. The creatinol sulfate of the creatinol compound may be able to be replaced by the chemical compounds selected from the group consisting of creatinol sulfate, creatinol hydroiodide, and creatinol hydrobromide to form the creatinol O-phosphate.

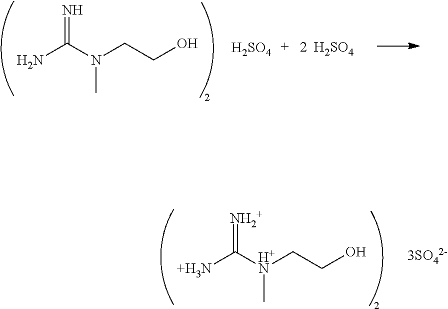

In a first example of the preferred embodiment of synthesizing creatinol O-phosphate by chemically reacting the P₂O₅ with the protected creatinol sulfate in step (2) to obtain the creatinol O-phosphate solution.

After the step (3) of crystallizing the creatinol O-phosphate solution to obtain the crystallized creatinol O-phosphate, a step of removing and rinsing the crystallized creatinol O-phosphate is preferably further provided for obtaining the relatively more pure crystallized creatinol O-phosphate, and a step of drying the rinsed crystallized creatinol to obtain the final product of the creatinol O-phosphate.

More specifically, in the first example, 20 g (0.06 mol) of protected creatinol sulfate is added into the reaction vessel, which is embodied as a 250 ml three-necked flask having a round-bottom shape. 150 ml of butanone is further added into the reaction vessel as the organic solvent, wherein the butanone and the protected creatinol sulfate are mechanically stirred for mixing therewith. The concentrated sulfuric acid is then being added into the reaction vessel to chemically react with the previously added reactants for 0.5 hour. After that, heat up the reactants in the reaction vessel to 45° C. to 50° C., and then add 9.5 g of P₂O₅. Keep the above step temperature for the reactants in the reaction vessel reacting 2 to 5 hours, and then add water 1.3 ml (0.07 mol) for reacting 0.5 to 2 hours. Filter and wash the product from the above reactions preferably via butanone until the eluate of the butanone is colorless. Dry the product to obtain the creatinol O-phosphate. The yield rate of the creatinol O-phosphate is about 65 to 90%.

Accordingly, the overall reaction equation of the creatinol sulfate reacted with the phosphorus pentoxide is shown as following:

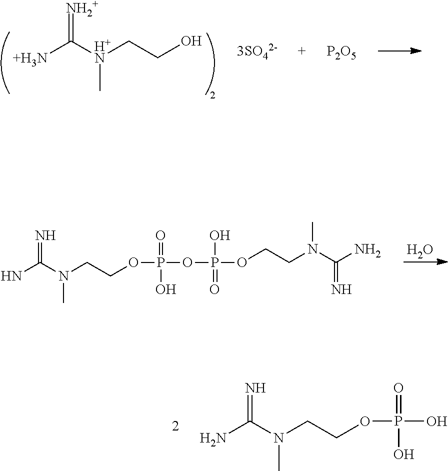

A second example of synthesis the creatinol O-phosphate according to the above preferred embodiment of the present invention is illustrated, wherein the creatinol O-phosphate is synthesized from the protected creatinol sulfate reacted with ClPO₃OH₂.

Accordingly, before the step (2), the process further comprises a step of chemically reacting POCl₃ with water to form the ClPO₃OH₂. Therefore, ClPO₃OH₂ can be chemically reacted with the protected creatinol sulfate to obtain a creatinol O-phosphate solution.

After the step (3) of crystallizing the creatinol O-phosphate solution to obtain the crystallized creatinol O-phosphate, a step of removing and rinsing the crystallized creatinol O-phosphate is preferably provided for obtaining the relatively more pure crystallized creatinol O-phosphate. Finally, another step of drying the rinsed crystallized creatinol O-phosphate to obtain the final product of the creatinol O-phosphate may be further provided after the step of removing and rinsing the crystallized creatinol O-phosphate to obtain the final product of the creatinol O-phosphate.

In order to specifically illustrating the process for producing the creatinol O-phosphate, the specific amounts and proportions of each chemical compounds involved in the reaction are illustrated in the second example of synthesizing the creatinol O-phosphate.

Accordingly, 18.5 g (0.12 mol) of POCl₃ is added into a 100 ml three-necked flask as the reaction container and then 4.3 ml (0.24 mol) of water is added therewithin to chemically react with the POCl₃ to form the ClPO₃OH₂. The temperature and time of the reaction is preferably controlled under 15° C. for 30 minutes.

20 g of the protected creatinol sulfate is taken for adding into a 500 ml three-necked flask of the reaction container and is mixed with 150 ml butanone as the organic solvent thereinto. Then, the above creatinol sulfate solution is stirred at room temperature for 1 to 2 hours. 12.2 g (0.12 mol) of the concentrated sulfuric acid is slowly added into the above creatinol sulfate solution for 30 minutes. After mixing the concentrated sulfuric acid, add the above prepared ClPO₃OH₂, and then keep stirring the above creatinol sulfate solution at the temperature of 30° C. to 40° C. for 1 to 4 hours.

Therefore, after the above reaction is done, the excessive sulfuric acid should be removed and other side products of the reaction may further conducted, wherein the undesired product may be removed by filtering the creatinol O-phosphate solution to obtain the crystallized creatinol O-phosphate, such as by vacuum filtration method to filter the solution. A butanone may be applied for washing the crystallized creatinol O-phosphate until an eluate of the butanone washed the crystallized creatinol O-phosphate is colorless. Finally, dry the washed crystallized creatinol O-phosphate to obtain the final product of the creatinol O-phosphate, which has a creatinol O-phosphate yield of 60% to 85% according to the second example.

Accordingly, the overall reaction equation of the creatinol sulfate reacted with the CIPO₃H₂ is shown as following:

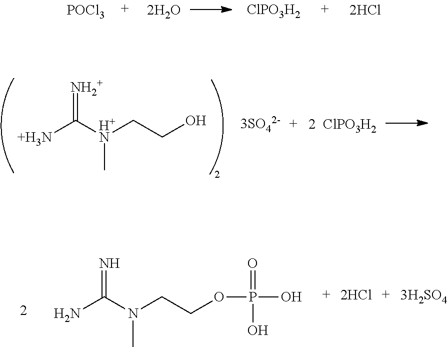

A third example of the synthesizing the creatinol O-phosphate according to the preferred embodiment of the present invention is illustrated, wherein in the third example, POCl₃ is adapted for chemically reacting with the protected creatinol sulfate to form the creatinol O-phosphate. Therefore, the step (2) of the synthesis process of the creatinol O-phosphate further comprises a step of (2.1) chemically reacting the POCl₃ with the creatinol sulfate to obtain a creatinol O-phosphate solution.

After the step (3) of crystallizing the creatinol O-phosphate solution to obtain the crystallized creatinol O-phosphate, a step of removing and rinsing the crystallized creatinol O-phosphate is preferably further provided for obtaining the relatively more pure crystallized creatinol O-phosphate, and a step of drying the rinsed crystallized creatinol to obtain the final product of the creatinol O-phosphate.

In order to specifically illustrating the process for producing the creatinol O-phosphate, the specific amounts and proportions of each chemical compounds involved in the reaction are illustrated in the third example of synthesizing the creatinol O-phosphate.

20 g (0.06 mol) of the creatinol sulfate is taken for adding into a 250 ml three-necked flask of the reaction container and mixed with 150 ml of butanone as the organic solvent, so as to form the creatinol sulfate solution. 12.2 g (0.12 mol) of the concentrated sulfuric acid is slowly added into the above creatinol sulfate solution while stirring thereof, and the reaction is controlled under 35° C. for 30 minutes.

In this example, 18.5 g (0.12 mol) of POCl₃ in the step (2) is added into the reactants under a temperature between 10° C. to 30° C. for 1 to 3 hours. Then add 4.3 ml (0.24 mol) of water into the above reactants for hydrolysis reaction for 0.5 to 1 hour.

Therefore, after the above reaction is done, a step after the step (2) of removing the excessive sulfuric acid and other side products of the reaction may further conducted, wherein the undesired product may be removed by filtering the creatinol O-phosphate solution to obtain the crystallized creatinol O-phosphate, such as by vacuum filtration method to filter the solution. A butanone may be applied for washing the crystallized creatinol O-phosphate until an eluate of the ethanol washed the crystallized creatinol O-phosphate is colorless. Finally, dry the washed crystallized to obtain the final product of the creatinol O-phosphate, which has a creatinol O-phosphate yield rate of 60% to 85% in the third example.

Accordingly, the overall reaction equation of the creatinol sulfate reacted with the phosphoryl chloride is shown as following:

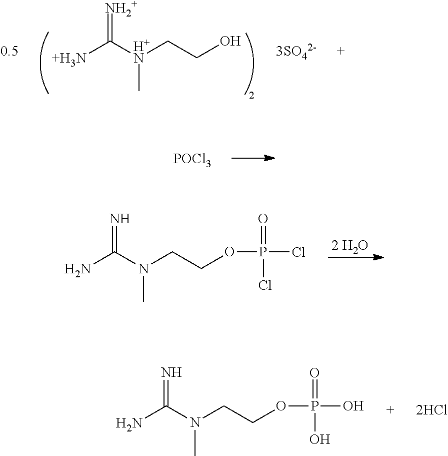

Accordingly, the above final products from the preferred process and its alternative processes may further comprises a step of re-crystallizing the final dried creatinol O-phosphate product to obtain a relatively higher purity of creatinol O-phosphate, which is about 99.05% purity of the creatinol O-phosphate after the re-crystallization.

Therefore, there is an example of the re-crystallization to exemplary illustrate the step of re-crystallizing. 5 g of the final product of the creatinol O-phosphate (about 92.55% purity of creatinol O-phosphate) is dissolved into a 30 to 50 ml water at the temperature between 50° C. to 70° C., and then filtering the above dissolved creatinol O-phosphate water solution while the solution is still warm. Cool down the filtered solution to room temperate for crystallizing for 2 to 4 hours. Then further vacuum filter the crystallizing creatinol O-phosphate dissolved into the water solution, which is re-crystallized creatinol O-phosphate having purity of 99.05%, and yield of 70% to 80%.

Accordingly, the melting point of the creatinol O-phosphate product is determined via melting point apparatus, which is around 243 to 244° C. The purity of the creatinol O-phosphate after re-crystallized is determined via High Performance Liquid Chromatography (HPLC). The chemical structure determination of the creatinol O-phosphate may be determined via hydrogen spectrometer and carbon spectrometer of nuclear magnetic resonance, H-NMR and C-NMR. The properties of creatinol O-phosphate may be determined by any other devices or method, which is able to analyze the creatinol O-phosphate of the final product.

Accordingly, the present invention also provides the method for synthesizing the creatinol O-phosphate, which is preferably formed from the creatinol sulfate chemically react with concentrated sulfuric acid or the likes to form the protected creatinol sulfate, so as to further react with POCl₃, ClPO₃OH₂, or P₂O₅ to form the creatinol O-phosphate. Therefore, the creatinol sulfate is adapted for forming the creatinol O-phosphate instead of using the S-ethyl isothiourea hydrobromide, which is high in price and generated relatively higher toxicity of sulfur alcohol, so that method provides relatively less toxic side products, so as to enhance the safety of the final product as being orally taken by human. The butanone using as the organic solvent has higher boiling point and lower volatile than acetone, which is commonly used in the existing process, so that less organic solvent of the butanone is required during the synthesis process. The entire reactions are completed under normal pressure, so that the energy consuming is reduced. The yield rate and purity of the creatinol O-phosphate is improved.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of producing a transitional compound for a synthesizing process of creatinol O-phosphate, comprising the step of adding sulfuric acid into a creatinol compound to form a protected functional group as a transition state of the synthesizing process of creatinol O-phosphate, wherein said creational compound has a chemical structure of:

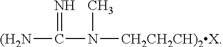

2. The method, as recited in claim 1, wherein said protected functional group is chemically reacting with a chemical compound selected from the group consisting of POCl₃, ClPO₃OH₂, and P₂O₅ to form said creatinol O-phosphate solution.

3. The method, as recited in claim 1, wherein the step comprises the sub-steps of:
(a) mixing said creatinol compound into an organic solvent to form a creatinol solution; and
(b) slowly dropping said sulfuric acid into said creatinol solution to form said protected functional group.

4. The method, as recited in claim 2, wherein the step comprises the sub-steps of:
(a) mixing said creatinol compound into an organic solvent to form a creatinol solution; and
(b) slowly dropping said sulfuric acid into said creatinol solution to form said protected functional group.

5. The method, as recited in claim 2, wherein said creatinol compound is creatinol sulfate such that when said creatinol sulfate is chemically reacted with sulfuric acid, a protected creatinol sulfate is produced as said protected functional group for providing said transition state of the synthesizing process of creatinol O-phosphate.

6. The method, as recited in claim 4, wherein said creatinol compound is creatinol sulfate such that when said creatinol sulfate is chemically reacted with sulfuric acid, a protected creatinol sulfate is produced as said protected functional group for providing said transition state of the synthesizing process of creatinol O-phosphate.

7. The method, as recited in claim 2, wherein said creatinol compound is selected from the group consisting of creatinol sulfate, creatinol hydroiodide, and creatinol hydrobromide to chemically react with sulfuric acid so as to form said protected functional group as said transition state of the synthesizing process of creatinol O-phosphate.

8. The method, as recited in claim 4, wherein said creatinol compound is selected from the group consisting of creatinol sulfate, creatinol hydroiodide, and creatinol hydrobromide to chemically react with sulfuric acid so as to form said protected functional group as said transition state of the synthesizing process of creatinol O-phosphate.

9. The method, as recited in claim 6, wherein said organic solvent in the step (a) is selected from the group consisting of butanone, ethyl acetate, acetone, and other ketones.

10. The method, as recited in claim 8, wherein said organic solvent in the step (a) is selected from the group consisting of butanone, ethyl acetate, acetone, and other ketones.

11. A transitional compound for a synthesizing process of creatinol O-phosphate, comprising a predetermined amount of sulfuric acid adding into a creatinol compound to form a protected functional group as a transition state of the synthesizing process of creatinol O-phosphate, wherein said creational compound has a chemical structure of:

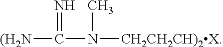

12. The transitional compound, as recited in claim 11, wherein said protected functional group is adapted for chemically reacting with a chemical compound selected from the group consisting of POCl₃, ClPO₃OH₂, and P₂O₅ to form said creatinol O-phosphate solution.

13. The transitional compound, as recited in claim 11, wherein said creatinol compound is initially mixed with an organic solvent to form a creatinol solution and then is chemically reacted said sulfuric acid with said creatinol solution to form said protected functional group.

14. The transitional compound, as recited in claim 12, wherein said creatinol compound is initially mixed with an organic solvent to form a creatinol solution and then is chemically reacted said sulfuric acid with said creatinol solution to form said protected functional group.

15. The transitional compound, as recited in claim 13, wherein said creatinol compound is creatinol sulfate such that when said creatinol sulfate is chemically reacted with sulfuric acid, a protected creatinol sulfate is produced as said protected functional group for providing said transition state of the synthesizing process of creatinol O-phosphate.

16. The transitional compound, as recited in claim 14, wherein said creatinol compound is creatinol sulfate such that when said creatinol sulfate is chemically reacted with sulfuric acid, a protected creatinol sulfate is produced as said protected functional group for providing said transition state of the synthesizing process of creatinol O-phosphate.

17. The transitional compound, as recited in claim 13, wherein said creatinol compound is selected from the group consisting of creatinol sulfate, creatinol hydroiodide, and creatinol hydrobromide to chemically react with sulfuric acid so as to form said protected functional group as said transition state of the synthesizing process of creatinol O-phosphate.

18. The transitional compound, as recited in claim 14, wherein said creatinol compound is selected from the group consisting of creatinol sulfate, creatinol hydroiodide, and creatinol hydrobromide to chemically react with sulfuric acid so as to form said protected functional group as said transition state of the synthesizing process of creatinol O-phosphate.

19. The transitional compound, as recited in claim 16, wherein said organic solvent in is selected from the group consisting of butanone, ethyl acetate, acetone, and other ketones.

20. The transitional compound, as recited in claim 18, wherein said organic solvent in is selected from the group consisting of butanone, ethyl acetate, acetone, and other ketones.
